(19) United States
(12) Patent Application Publication
(10) Pub. No.: US 2023/0192856 A1
(43) Pub. Date: Jun. 22, 2023

(54) DOSING AND ADMINISTRATION OF ACTIVATABLE ANTI-CTLA-4 ANTIBODY

(71) Applicant: BRISTOL-MYERS SQUIBB COMPANY, Princeton, NJ (US)

(72) Inventors: Leonard P. JAMES, Princeton, NJ (US); Michelle BROWN, Yardley, PA (US)

(21) Appl. No.: 17/998,521

(22) PCT Filed: May 11, 2021
(86) PCT No.: PCT/US2021/031670

§ 371 Date: Nov. 11, 2022

Related U.S. Application Data
(60) Provisional application No. 63/023,850, filed on May 12, 2020.

(51) Int. Cl. C07K 16/28

(52) U.S. Cl. CPC C07K 16/2818; C07K 2317/21; C07K 2317/565

(57) ABSTRACT

The present invention provides methods of dosing and administration of an activatable anti-CTLA-4 antibody, such as an activatable ipilimumab.

### CROSS-REFERENCE TO RELATED APPLICATIONS

This application claims the benefit under 35 U.S.C. §119(e) of US Provisional Application Ser. No. 63/023,850, filed May 12, 2020; the disclosure of which is incorporated herein by reference.

### SEQUENCE LISTING

The Sequence Listing filed electronically herewith is also hereby incorporated by reference in its entirety (File Name: 20210421_SEQL_13580WOPCT_GB.txt; Date Created: 21 Apr. 2021; File Size: 38 KB).

### FIELD OF THE INVENTION

The present application discloses methods of dosing and administration of activatable anti-CTLA-4 antibodies for treating cancer.

### BACKGROUND OF THE INVENTION

The immune system is capable of controlling tumor development and mediating tumor regression. This requires the generation and activation of tumor antigen-specific T cells. Multiple T-cell co-stimulatory receptors and T-cell negative regulators, or co-inhibitory receptors, act in concert to control T-cell activation, proliferation, and gain or loss of effector function. Among the earliest and best characterized T-cell co-stimulatory and co-inhibitory molecules are CD28 and CTLA-4. Rudd et al. (2009) Immunol. Rev. 229: 12. CD28 provides co-stimulatory signals to T-cell receptor engagement by binding to B7-1 and B7-2 ligands on antigen-presenting cells, while CTLA-4 provides a negative signal down-regulating T-cell proliferation and function. CTLA-4, which also binds the B7-1 (CD80) and B7-2 (CD86) ligands but with higher affinity than CD28, acts as a negative regulator of T-cell function through both cell autonomous (or intrinsic) and cell non-autonomous (or extrinsic) pathways. Intrinsic control of CD8 and CD4 T effector (T_{eff}) function is mediated by the inducible surface expression of CTLA-4 as a result of T-cell activation, and inhibition of T-cell proliferation and cytokine production by multivalent engagement of B7 ligands on opposing cells. Peggs et al. (2008) Immunol. Rev. 224:141.

Anti-CTLA-4 antibodies, when cross-linked, suppress T cell function in vitro. Krummel & Allison (1995) J. Exp. Med. 182:459; Walunas et al. (1994) Immunity 1:405. Regulatory T cells (T_{regs}), which express CTLA-4 constitutively, control effector T cell (T_{eff}) function in a non-cell autonomous fashion. T_{regs} that are deficient for CTLA-4 have impaired suppressive ability (Wing et al. (2008) Science 322:271) and antibodies that block CTLA-4 interaction with B7 can inhibit T_{reg} function (Read et al. (2000) J. Exp. Med. 192:295; Quezada et al. (2006) J. Clin. Invest. 116:1935). More recently, T_{effs} have also been shown to control T cell function through extrinsic pathways (Corse & Allison (2012) J. Immunol. 189:1123; Wang et al. (2012) J. Immunol. 189:1118). Extrinsic control of T cell function by T_{regs} and T_{effs} occurs through the ability of CTLA-4-positive cells to remove B7 ligands on antigen-presenting cells, thereby limiting their co-stimulatory potential. Qureshi et al. (2011) Science 332: 600; Onishi et al. (2008) Proc. Nat′l Acad. Sci. (USA) 105:10113. Antibody blockade of CTLA-4/B7 interactions is thought to promote T_{eff} activation by interfering with negative signals transmitted by CTLA-4 engagement; this intrinsic control of T-cell activation and proliferation can promote both T_{eff} and T_{reg} proliferation (Krummel & Allison (1995) J. Exp. Med. 182:459; Quezada et al. (2006) J. Clin. Invest. 116:1935). In early studies with animal models, antibody blockade of CTLA-4 was shown to exacerbate autoimmunity. Perrin et al. (1996) J. Immunol. 157:1333; Hurwitz et al. (1997) J. Neuroimmunol. 73:57. By extension to tumor immunity, the ability of anti-CTLA-4 to cause regression of established tumors provided a dramatic example of the therapeutic potential of CTLA-4 blockade. Leach et al. (1996) Science 271:1734.

Human antibodies to human CTLA-4, ipilimumab and tremelimumab, were selected to inhibit CTLA-4-B7 interactions (Keler et al. (2003) J. Immunol. 171:6251; Ribas et al. (2007) Oncologist 12:873) and have been tested in a variety of clinical trials for multiple malignancies. Hoos et al. (2010) Semin. Oncol. 37:533; Ascierto et al. (2011) J. Transl. Med. 9:196. Ipilimumab, which was first approved for the treatment of metastatic melanoma, has since been approved for use in other cancers, and is in clinical testing in yet other cancers. Hoos et al. (2010) Semin. Oncol. 37:533; Hodi et al. (2010) N. Engl. J. Med. 363:711; Pardoll (2012) Nat. Immunol. 13(12): 1129. In 2011, ipilimumab, which has an IgG1 constant region, was approved in the US and EU for the treatment of unresectable or metastatic melanoma based on an improvement in overall survival in a phase III trial of previously treated patients with advanced melanoma. Hodi et al. (2010) N. Engl. J. Med. 363:711. Tumor regressions and disease stabilization were frequently observed, but treatment with these antibodies has been accompanied by adverse events with inflammatory infiltrates capable of affecting a variety of organ systems. The severity and frequency of side effects from treatment with ipilimumab, which carries a black box warning of immune-mediated adverse reactions, and to an even greater extent when combined with nivolumab (OPDIVO®), limits the use of ipilimumab by many treating physicians.

Activatable forms of ipilimumab have been developed in which the light chain contains a masking moiety that interferes with binding to CTLA-4, but is released preferentially in the tumor microenvironment after cleavage by proteases that are more prevalent and/or active in tumors than in peripheral tissues. WO 18/085555. Such tumor-specific activation enables full CTLA-4 blocking activity in the tumor microenvironment, promoting anti-tumor immune response, while minimizing CTLA-4 blockade in normal tissue, where it could otherwise cause systemic toxicity. Thereby the activatable form results is an increased therapeutic index compared with the native parent molecule.

Although the novel mechanism of action of activatable CTLA-4 antibodies provides therapeutic benefits, it presents challenges with regard to methods of dosing and administration due to novel pharmacokinetic and pharmacodynamic considerations not present in treatment with ipilimumab. Known methods for dosing and administration of ipilimumab may therefore be inapplicable to treatment with activatable CTLA-4 antibodies. The need exists for methods of dosing and administration of activatable anti-CTLA-4 antibodies, such as Activatable Ipilimumab, that maximize its therapeutic index and optimize the exposure to activated ipilimumab.

### SUMMARY OF THE INVENTION

The present invention provides methods of dosing and administration of an activatable anti-CTLA-4 antibody in which the antibody is administered as monotherapy once every four weeks (Q4W) or once every eight weeks (Q8W). The invention further provides methods of dosing and administration of an activatable anti-CTLA-4 antibody in combination with an anti-PD1 or anti-PD-L1 antibody, such as nivolumab, in which the activatable anti-CTLA-4 antibody is administered once every four weeks (Q4W) or once every eight weeks (Q8W).

In some embodiments the activatable anti-CTLA-4 antibody is an activatable form of ipilimumab, such as an antibody comprising a heavy chain comprising the heavy chain variable region sequence of SEQ ID NO: 9 and a light chain comprising a light chain variable region sequence selected from the group consisting of SEQ ID NOs: 21, 22 and 23 (“Activatable Ipilimumab”).

In various embodiments, the activatable anti-CTLA-4 antibody, such as Activatable Ipilimumab, is administered as monotherapy at a flat dose of 240, 800, 1600 or 2400 mg. In one embodiment, the activatable anti-CTLA-4 antibody is administered at 1600 mg, and may optionally be administered Q8W.

In additional embodiments, the activatable anti-CTLA-4 antibody is administered in combination with an anti-PD-1 or anti-PD-L1 antibody, such as nivolumab, at a flat dose of 240, 600, 800, 1200, or 1600 mg. In various combination embodiments, the anti-PD-1 or anti-PD-L1 antibody, such as nivolumab, is administered at a flat dose of 160, 360 or 480 mg.

In one combination therapy embodiment, the activatable anti-CTLA-4 antibody is administered at a flat dose of 240 mg and anti-PD-1 or anti-PD-L1 antibody is administered at a flat dose of 360 mg, both Q3W. In a further embodiment, the preceding combination therapy is administered for four courses of treatment, followed by maintenance treatment with 360 mg nivolumab Q4W continuously.

In one combination therapy embodiment, the activatable anti-CTLA-4 antibody is administered at a flat dose of 800 mg Q8W and anti-PD-1 or anti-PD-L1 antibody is administered at a flat dose of 480 mg Q4W. In another combination therapy embodiment, the activatable anti-CTLA-4 antibody is administered at a flat dose of 1200 mg Q8W and anti-PD-1 or anti-PD-L1 antibody is administered at a flat dose of 480 mg Q4W. In a specific embodiment, Activatable Ipilimumab is administered at a flat dose of 1200 mg Q8W and nivolumab is administered at a flat dose of 480 mg Q4W.

In another combination therapy embodiment, the activatable anti-CTLA-4 antibody is administered at a flat dose of 600 mg Q4W and anti-PD-1 or anti-PD-L1 antibody is administered at a flat dose of 480 mg Q4W. In selected combination therapy embodiments the activatable anti-CTLA-4 antibody is Activatable Ipilimumab and the anti-PD-1 or anti-PD-L1 antibody is nivolumab. In a specific embodiment, Activatable Ipilimumab is administered at a flat dose of 600 mg Q4W and nivolumab is administered at a flat dose of 480 mg Q4W.

In some embodiments, unit doses of the therapeutic antibodies of the present invention are packaged in a format selected from the group consisting of vials, ampules, prefilled syringes and autoinjectors.

In some embodiments, Activatable Ipilimumab, as used herein, refers to an activatable form of ipilimumab comprising a heavy chain comprising the heavy chain variable region of SEQ ID NO: 9 and a light chain comprising a light chain variable region sequence selected from the group consisting of SEQ ID NOs: 21, 22 and 23. The light chain variable domain of an Activatable Ipilimumab may optionally further comprise a spacer of SEQ ID NO: 16 and the light chain may comprise a kappa constant domain of SEQ ID NO: 14, for example the spacer YV39-2011 light chain provided at SEQ ID NO: 24. The heavy chain of an Activatable Ipilimumab may further comprise an IgG1 constant domain of SEQ ID NO: 10, for example as in the ipilimumab heavy chain provided at SEQ ID NO: 11 or 12. Activatable Ipilimumab may comprise a heavy chain comprising SEQ ID NO: 11 or 12 and a light chain comprising a light chain of SEQ ID NO: 24.

In various embodiments, the anti-PD1 of anti-PD-L1 is nivolumab comprising the heavy chain sequence of SEQ ID NO: 25 or 26 and the light chain sequence of SEQ ID NO: 27.

The methods of dosing and administration of the present invention may be used to treat various diseases, such as cancers, including small-cell lung cancer (SCLC), non-small cell lung cancer (NSCLC), colorectal cancer (CRC), renal cell carcinoma (RCC), castrate-resistant prostate cancer (CRPC), bladder cancer, gastric cancer, esophageal cancer, and melanoma. In specific embodiments, the methods of dosing and administration of the present invention are used for the treatment indications for which ipilimumab is approved, such as unresectable or metastatic melanoma, or adjuvant treatment of melanoma, or when administered in combination with an anti-PD1 or anti-PD-L1 antibody, such as nivolumab, advanced renal cell carcinoma (RCC), microsatellite instability-high (MSI-H) or mismatch repair deficient (dMMR) metastatic colorectal cancer, melanoma, non-small cell lung cancer (NSCLC), malignant pleural mesothelioma, or hepatocellular carcinoma. In one embodiment, the methods of dosing and administration of the present invention are used to treat previously untreated unresectable stage III-IV melanoma.

### DETAILED DESCRIPTION OF THE INVENTION

### Definitions

In order that the present disclosure may be more readily understood, certain terms are first defined. As used in this application, except as otherwise expressly provided herein, each of the following terms shall have the meaning set forth below. Additional definitions are set forth throughout the application.

“Activatable anti-CTLA-4 antibodies,” as used herein, refers to modified forms of antagonist anti-CTLA-4 antibodies that block binding of CTLA-4 to B7 ligands, that comprise structural modifications that inhibit binding to CTLA-4 until cleaved by proteases more prevalent and/or active in the tumor microenvironment. “Activatable anti-CTLA-4 antibodies” encompasses activatable forms of ipilimumab, such as antibodies comprising light chains modified to comprise a masking moiety (MM) and a cleavable moiety (CM), as disclosed in WO 18/085555, for example, Activatable Ipilimumab.

“Activatable Ipilimumab,” as used herein, refers to an activatable form of ipilimumab comprising a heavy chain comprising the heavy chain variable region sequence of SEQ ID NO: 9 and a light chain comprising a light chain variable region sequence selected from the group consisting of SEQ ID NOs: 21, 22 and 23. The light chain variable domain of an Activatable Ipilimumab may optionally further comprise a spacer of SEQ ID NO: 16 and the light chain may comprise a kappa constant domain of SEQ ID NO: 14, for example the spacer YV39-2011 light chain provided at SEQ ID NO: 24. The heavy chain of an Activatable Ipilimumab may further comprise an IgG1 constant domain of SEQ ID NO: 10, for example as in the ipilimumab heavy chain provided at SEQ ID NO: 11 or 12. Activatable Ipilimumab may comprise a heavy chain comprising SEQ ID NO: 11 or 12 and a light chain comprising a light chain of SEQ ID NO: 24.

“Adjuvant,” as used herein, refers to an agent that is administered to a subject in conjunction with a vaccine to enhance the immune response to the vaccine compared with the immune response that would result from administration of the vaccine without the adjuvant. Adjuvant may also refer to use of an agent after surgical removal of a tumor to reduce the risk of disease recurrence, such as use of ipilimumab or Activatable Ipilimumab following surgical removal of a melanoma.

“Administering,” “administer” or “administration” refers to the physical introduction of a composition comprising a therapeutic agent to a subject, using any of the various methods and delivery systems known to those skilled in the art. Preferred routes of administration for antibodies of the invention include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrastemal injection and infusion, as well as in vivo electroporation. Alternatively, an antibody of the invention can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

Unless otherwise indicated, administration of antibodies for the treatment of cancer is parenteral, such as intravenous (iv) or subcutaneous (sc). Methods of dosing and administration of the present invention can be performed for any number of cycles of treatment, from one, two, three, four cycles, etc., up to continuous treatment (repeating the dosing until no longer necessary, disease recurrence, or unacceptable toxicity is reached). For the purposes of combination therapy embodiments of the present disclosure, one cycle comprises the minimal unit of administration that includes at least one dose of each component (drug).

“Initial Dose” or “initial dosing” as used herein refers to the first dosing of a patient with the regimen, and any subsequent repetitions of that same dosing regimen (such as second, third and fourth cycles, etc.), and is contrasted with “maintenance dose” or “maintenance dosing,” which refers to subsequent doses administered over a longer period after the initial dose or doses, e.g. longer than three months up to several years, or even indefinitely. Maintenance dosing may optionally comprise less frequent dosing and/or lower dose than the initial dose. Unless otherwise indicated, the dosing regimens disclosed and claimed herein constitute initial doses and initial dosing.

“Combination therapy,” as used herein, refers to administration of two or more therapeutic agents in a coordinated treatment plan, in which the dose and dosing interval of a first component of the combination is based on the dose and dosing interval of a second component, to elicit an overall therapeutic benefit. It is not limited to any particular details of administration, and encompasses administration as a mixture of the components, administration as separate compositions, whether concurrent or sequential on a given day. Although combination therapy is most convenient when dosing schedules are the same or multiples of one another (e.g. Q4W and Q8W), it also encompasses administration on different days if dosing intervals do not align for any given cycle.

An “antibody” (Ab) shall include, without limitation, a glycoprotein immunoglobulin which binds specifically to an antigen and comprises at least two heavy chains (HC) and two light chains (LC) interconnected by disulfide bonds. Each heavy chain comprises a heavy chain variable region (abbreviated herein as V_{H}) and a heavy chain constant region. The heavy chain constant region comprises three domains, C_{H1}, C_{H2} and C_{H3}. Each light chain comprises a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen.

As used herein, and in accord with conventional interpretation, an antibody that is described as comprising “a” heavy chain and/or “a” light chain refers to antibodies that comprise “at least one” of the recited heavy and/or light chains, and thus will encompass antibodies having two or more heavy and/or light chains. Specifically, antibodies so described will encompass conventional antibodies having two substantially identical heavy chains and two substantially identical light chains. Antibody chains may be substantially identical but not entirely identical if they differ due to post-translational modifications, such as C-terminal cleavage of lysine residues, alternative glycosylation patterns, etc. Antibodies differing in fucosylation within the glycan, however, are not substantially identical.

As used herein, the “light chain variable domain” of an antibody light chain comprises the light chain framework regions (FR) and CDR sequences, such as FR1-CDRL1-FR2-CDRL2-FR3-CDRL3-FR4, such as the light chain variable domain of ipilimumab as provided at SEQ ID NO: 13. When used with reference to activatable anti-CTLA-4 antibodies, the “light chain variable domain” may further comprise a masking moiety, a cleavable moiety, and optionally other sequence elements as disclosed herein.

Unless indicated otherwise or clear from the context, an antibody defined by its target specificity (e.g. an “anti-CTLA-4 antibody”) refers to antibodies that can bind to its human target (i.e. human CTLA-4). Such antibodies may or may not bind to CTLA-4 from other species.

The immunoglobulin may derive from any of the commonly known isotypes, including but not limited to IgA, secretory IgA, IgG and IgM. The IgG isotype may be divided in subclasses in certain species: IgG1, IgG2, IgG3 and IgG4 in humans, and IgG1, IgG2a, IgG2b and IgG3 in mice. “Isotype” refers to the antibody class (e.g., IgM or IgG1) that is encoded by the heavy chain constant region genes. “Antibody” includes, by way of example, both naturally occurring and non-naturally occurring antibodies, including allotypic variants; monoclonal and polyclonal antibodies; chimeric and humanized antibodies; human or non-human antibodies; wholly synthetic antibodies; and single chain antibodies. Unless otherwise indicated, or clear from the context, antibodies disclosed herein are human IgG1 antibodies. IgG1 constant domain sequences include, but are not limited to, known IgG1 allotypic variants. Sequences in the Sequence Listing, of course, comprise the sequences provided and not any other sequences.

An “isolated antibody” refers to an antibody that is substantially free of other antibodies having different antigenic specificities (e.g., an isolated antibody that binds specifically to CTLA-4 is substantially free of antibodies that bind specifically to antigens other than CTLA-4). An isolated antibody that binds specifically to CTLA-4 may, however, cross-react with other antigens, such as CTLA-4 molecules from different species. Moreover, an isolated antibody may be substantially free of other cellular material and/or chemicals. By comparison, an “isolated” nucleic acid refers to a nucleic acid composition of matter that is markedly different, i.e., has a distinctive chemical identity, nature and utility, from nucleic acids as they exist in nature. For example, an isolated DNA, unlike native DNA, is a free-standing portion of a native DNA and not an integral part of a larger structural complex, the chromosome, found in nature. Further, an isolated DNA, unlike native DNA, can be used as a PCR primer or a hybridization probe for, among other things, measuring gene expression and detecting biomarker genes or mutations for diagnosing disease or predicting the efficacy of a therapeutic. An isolated nucleic acid may also be purified so as to be substantially free of other cellular components or other contaminants, e.g., other cellular nucleic acids or proteins, using standard techniques well known in the art.

The term “monoclonal antibody” (“mAb”) refers to a preparation of antibody molecules of single molecular composition, i.e., antibody molecules whose primary sequences are essentially identical, and which exhibit a single binding specificity and affinity for a particular epitope. Monoclonal antibodies may be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

An “antibody fragment” refers to a portion of a whole antibody, generally including the “antigen-binding portion” (“antigen-binding fragment”) of an intact antibody which retains the ability to bind specifically to the antigen bound by the intact antibody and also retains the Fc region of an antibody mediating FcR binding capability.

“Antibody-dependent cell-mediated cytotoxicity” (“ADCC”) refers to an in vitro or in vivo cell-mediated reaction in which nonspecific cytotoxic cells that express FcRs (e.g., natural killer (NK) cells, macrophages, neutrophils and eosinophils) recognize antibody bound to a surface antigen on a target cell and subsequently cause lysis of the target cell. In principle, any effector cell with an activating FcR can be triggered to mediate ADCC.

“Cancer” refers a broad group of various diseases characterized by the uncontrolled growth of abnormal cells in the body. Unregulated cell division and growth divide and grow results in the formation of malignant tumors or cells that invade neighboring tissues and may also metastasize to distant parts of the body through the lymphatic system or bloodstream.

A “cell surface receptor” refers to molecules and complexes of molecules capable of receiving a signal and transmitting such a signal across the plasma membrane of a cell.

“Effector function” refers to the interaction of an antibody Fc region with an Fc receptor or ligand, or a biochemical event that results therefrom. Exemplary “effector functions” include Clq binding, complement dependent cytotoxicity (CDC), Fc receptor binding, FcyR-mediated effector functions such as ADCC and antibody dependent cell-mediated phagocytosis (ADCP), and down-regulation of a cell surface receptor (e.g., the B cell receptor; BCR). Such effector functions generally require the Fc region to be combined with a binding domain (e.g., an antibody variable domain).

An “immune response” refers to a biological response within a vertebrate against foreign agents, which response protects the organism against these agents and diseases caused by them. The immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate’s body of invading pathogens, cells or tissues infected with pathogens, cancerous or other abnormal cells, or, in cases of autoimmunity or pathological inflammation, normal human cells or tissues.

An “immunomodulator” or “immunoregulator” refers to a component of a signaling pathway that may be involved in modulating, regulating, or modifying an immune response. “Modulating,” “regulating,” or “modifying” an immune response refers to any alteration in a cell of the immune system or in the activity of such cell. Such modulation includes stimulation or suppression of the immune system which may be manifested by an increase or decrease in the number of various cell types, an increase or decrease in the activity of these cells, or any other changes which can occur within the immune system. Both inhibitory and stimulatory immunomodulators have been identified, some of which may have enhanced function in a tumor microenvironment. In preferred embodiments of the disclosed invention, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying an immune response.

“Potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

A “protein” refers to a chain comprising at least two consecutively linked amino acid residues, with no upper limit on the length of the chain. One or more amino acid residues in the protein may contain a modification such as, but not limited to, glycosylation, phosphorylation or disulfide bond formation. The term “protein” is used interchangeable herein with “polypeptide.”

A “subject” includes any human or non-human animal. The term “non-human animal” includes, but is not limited to, vertebrates such as nonhuman primates, sheep, dogs, rabbits, rodents such as mice, rats and guinea pigs, avian species such as chickens, amphibians, and reptiles. In preferred embodiments, the subject is a mammal such as a nonhuman primate, sheep, dog, cat, rabbit, ferret or rodent. In more preferred embodiments of any aspect of the disclosed invention, the subject is a human. Unless otherwise indicated, a subject as referred to herein is a human. The terms “subject” and “patient” are used interchangeably herein.

A “therapeutically effective amount” or “therapeutically effective dosage” of a drug or therapeutic agent, such as an Fc fusion protein of the invention, is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A therapeutically effective amount or dosage of a drug includes a “prophylactically effective amount” or a “prophylactically effective dosage,” which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent promotes cancer regression in a subject. In preferred embodiments, a therapeutically effective amount of the drug promotes cancer regression to the point of eliminating the cancer. “Promoting cancer regression” means that administering an effective amount of the drug, alone or in combination with an antineoplastic agent, results in a reduction in tumor growth or size, necrosis of the tumor, a decrease in severity of at least one disease symptom, an increase in frequency and duration of disease symptom-free periods, a prevention of impairment or disability due to the disease affliction, or otherwise amelioration of disease symptoms in the patient. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount or dosage of the drug preferably inhibits cell growth or tumor growth by at least about 20%, more preferably by at least about 40%, even more preferably by at least about 60%, and still more preferably by at least about 80% relative to untreated subjects. In the most preferred embodiments, a therapeutically effective amount or dosage of the drug completely inhibits cell growth or tumor growth, i.e., preferably inhibits cell growth or tumor growth by 100%. The ability of a compound to inhibit tumor growth can be evaluated in an animal model system, such as the CT26 colon adenocarcinoma, MC38 colon adenocarcinoma and Sa1N fibrosarcoma mouse tumor models, which are predictive of efficacy in human tumors. Alternatively, this property of a composition can be evaluated by examining the ability of the compound to inhibit cell growth, such inhibition can be measured in vitro by assays known to the skilled practitioner. In other preferred embodiments of the invention, tumor regression may be observed and continue for a period of at least about 20 days, more preferably at least about 40 days, or even more preferably at least about 60 days.

“Treatment” or “therapy” of a subject refers to any type of intervention or process performed on, or administering an active agent to, the subject with the objective of reversing, alleviating, ameliorating, inhibiting, slowing down or prevent the onset, progression, development, severity or recurrence of a symptom, complication, condition or biochemical indicia associated with a disease.

### Dosing and Administration of Activatable Anti-CTLA-4 Antibodies

The only approved anti-CTLA-4 antibody, ipilimumab (YERVOY®), provides long-term survival in up to 25% of metastatic melanoma patients when administered at 3 mg/kg (metastatic melanoma) or 10 mg/kg (adjuvant melanoma), but treatment is often accompanied by toxicity. Activatable antibodies that are preferentially activated by tumor-associated proteases hold the promise of reducing peripheral toxicity at a given dose, allowing higher (and thus potentially more efficacious) doses for any given level of toxicity, or some intermediate tread-off of the two. Activatable Ipilimumab has been proposed as an improved, safer way to target the CTLA-4 pathway than ipilimumab, which is known to cause limiting side-effects at higher doses. WO 18/085555. The methods of dosing and administration provided herein are essential to get the greatest benefit from the activatable antibody approach, and maximize the therapeutic index. The novel mechanism of action of tumor-activatable anti-CTLA-4 antibody treatment means that there is no prior dosing data and experience to rely on.

The present invention is based in part on results of early human clinical trials of Activatable Ipilimumab. Analysis of tumor biopsies demonstrated that, as intended, Activatable Ipilimumab is preferentially converted to mono- and dual-cleaved forms within the TME as compared to the plasma. This preferential cleavage leads to improved safety, since peripheral anti-CTLA-4 activity (cleaved species) is lower for any given level of anti-CTLA-4 activity within the tumor.

In addition, gene expression studies on biopsies of subjects treated with Activatable Ipilimumab showed the same pattern of results seen previously with ipilimumab, suggesting that the effects of Activatable Ipilimumab are mediated by the same anti-CTLA-4 activity as ipilimumab, consistent with the expected mechanism of action. Subjects showing a clinical benefit (shrinkage of a target lesion) from treatment with Activatable Ipilimumab exhibit elevated expression of a panel of inflammatory genes on day 15 of their first treatment cycle compared to expression before treatment. In contrast, neither subjects with stable disease (<20% growth, but not shrinkage, of a target lesion) nor those showing no clinical benefit (>20% growth target lesion) exhibited this elevated expression of inflammatory genes. The same pattern was observed in clinical trials of ipilimumab.

In addition, dosing studies with Activatable Ipilimumab surprisingly showed that Q8W dosing of Activatable Ipilimumab is superior to Q4W dosing. PK studies of the first cycle revealed that subjects treated at 1600 mg Q8W exhibited equivalent Cmax exposure to mono- and dual-cleaved species as subjects treated with 1600 mg Q4W. Subjects treated with 1600 mg Q8W also showed approximately twice the exposure to mono- and dual-cleaved species as those treated with 800 mg Q4W. Q8W was also found to be safer than dosing with the same amount of drug Q4W. Subjects administered 1600 mg Q8W had lower frequency of adverse events than subjects treated with 800 mg Q4W. These results taken together suggest that Q8W dosing of Activatable Ipilimumab is superior to Q4W dosing, providing enhanced exposure for a given amount of drug administered, and with significantly lower side effects. This improved profile may be due in part to the kinetics of exposure to the activated compound, because the active drug is generated continuously from the parent compound and thus has a longer apparent half-life.

Nevertheless, Q4W dosing of Activatable Ipilimumab with anti-PD-1 or anti-PD-L1 antibody, such as nivolumab, remains a viable alternative combination therapy dosing schedule. Such Q4W dosing aligns this combination therapy regimen with the Q2W or Q4W dosing schedule used with nivolumab (OPDIVO®) monotherapy, and thus is more convenient and less expensive than the existing approved Q3W combination therapy regimens for YERVOY® and OPDIVO®. OPDIVO® Prescribing Information, updated March 2020.

Therapeutic antibodies for treatment of cancer are typically administered at intervals approximating the half-life of the antibody in human subjects, which is approximately 21 days for an IgG. Currently approved monoclonal antibodies for treatment of cancer are typically dosed every one (QW), two (Q2W), three (Q3W) or four weeks (Q4W), with Q2W and Q3W being most common. Hendrikx et al. (2017) Oncologist 22:1212, Ovacik and Lin (2018) Clin. Transl. Sci. 11:540. For example, various approved therapeutic antibodies for treatment of cancer are administered Q2W/Q4W (OPDIVO®); Q3W (KEYTRUDA®; YERVOY®); Q2W/Q3W/Q4W (TECENTRIQ®). The half-life of YERVOY® (ipilimumab) is 15.4 days, and it is approved for administration Q3W, although it is also administered Q12W for long term maintenance for adjuvant melanoma use following an initial fours doses Q3W. YERVOY Prescribing Information, updated March 2020. Dosing intervals approximating the antibody half-life are rational in that they ensure replenishment before drug levels drop significantly, thus promoting a uniform circulating drug level (exposure) at steady state.

Activatable Ipilimumab comprises two heavy chains and two light chains in a conventional bivalent IgG structure, albeit with additional sequence elements (including MM and CM) at the amino termini of the light chains. Since each CM can be cleaved independently, Activatable Ipilimumab can exist in intact/uncleaved, mono-cleaved, and dual-cleaved forms all at the same time. Without intending to be limited by theory, the surprising results regarding Q8W dosing may be a consequence of the complex pharmacokinetics of these three distinct antibody species, in which mono- and dual-cleaved species are created from intact Activatable Ipilimumab over time by protease cleavage, while at the same time the levels of all species decay with their own unique half-lives. Without intending to be limited by theory, Q8W dosing may prevent build-up of mono- and dual-cleaved (active) species in the periphery, which might otherwise occur (as with Q4W dosing) and cause side effects.

TABLE 1

Summary of the Sequence Listing

| SEQ ID NO. | Description |
|---|---|
| 1 | human CTLA-4 (NP 005205.2) |
| 2 | human CD28 (NP 006130.1) |
| 3 | ipilimumab CDRH1 |
| 4 | ipilimumab CDRH2 |
| 5 | ipilimumab CDRH3 |
| 6 | ipilimumab CDRL1 |
| 7 | ipilimumab CDRL2 |
| 8 | ipilimumab CDRL3 |
| 9 | ipilimumab heavy chain variable domain |
| 10 | IgG1 constant domain |
| 11 | ipilimumab heavy chain lacking C-terminal K |
| 12 | ipilimumab heavy chain |
| 13 | ipilimumab light chain variable domain |
| 14 | kappa constant domain |
| 15 | ipilimumab light chain |
| 16 | Spacer QGQSGS |
| 17 | masking moiety YV39 |
| 18 | cleavable moiety 2001 |
| 19 | cleavable moiety 2011 |
| 20 | cleavable moiety 2012 |
| 21 | YV39-2001 VL |
| 22 | YV39-2011 VL |
| 23 | YV39-2012 VL |
| 24 | Spacer YV39-2011 LC |
| 25 | nivolumab heavy chain lacking C-terminal K |
| 26 | nivolumab heavy chain |
| 27 | nivolumab light chain |

With regard to antibody sequences, the Sequence Listing provides the sequences of the mature variable regions and heavy and light chains, i.e. the sequences do not include signal peptides.

### EQUIVALENTS

Those skilled in the art will recognize, or be able to ascertain using no more than routine experimentation, many equivalents of the specific embodiments disclosed herein. Such equivalents are intended to be encompassed by the following claims.

1. A method of treating a cancer in a human subject in need thereof with an activatable anti-CTLA-4 antibody, the method comprising administering the activatable anti-CTLA-4 antibody once every four weeks (Q4W) or once every eight weeks (Q8W);
wherein the activatable anti-CTLA-4 antibody comprises:
a. a heavy chain comprising the heavy chain variable domain sequence of SEQ ID NO: 9; and
b. a light chain comprising a light chain variable domain sequence selected from the group consisting of SEQ ID NOs: 21, 22 and 23.

2. The method of claim 1 wherein the activatable anti-CTLA-4 antibody comprises:
a. a heavy chain comprising the sequence of SEQ ID NO: 11; and
b. a light chain comprising the sequence of SEQ ID NO. 24.

3. The method of claim 1 wherein the activatable anti-CTLA-4 antibody is administered Q8W.

4. The method of claim 1 wherein the activatable anti-CTLA-4 antibody is administered at a flat dose of 240, 800, 1600 or 2400 mg.

5. The method of claim 4 wherein the activatable anti-CTLA-4 antibody is administered at a flat dose of 1600 mg Q8W.

6. The method of claim 1 wherein the cancer is selected from the group consisting of:
a. unresectable or metastatic melanoma; and
b. adjuvant treatment of melanoma.

7. A method of treating a cancer in a human subject in need thereof with an activatable anti-CTLA-4 antibody in combination with nivolumab, comprising administering the activatable anti-CTLA-4 antibody once every four weeks (Q4W) or once every eight weeks (Q8W), wherein the activatable anti-CTLA-4 antibody comprises:
a. a heavy chain comprising the heavy chain variable domain sequence of SEQ ID NO: 9; and
b. a light chain comprising a light chain variable domain sequence selected from the group consisting of SEQ ID NOs: 21, 22 and 23.

8. The method of claim 7 wherein the activatable anti-CTLA-4 antibody comprises:
a. a heavy chain comprising the sequence of SEQ ID NO: 11; and
b. a light chain comprising the sequence of SEQ ID NO. 24.

9. The method of claim 7, wherein the activatable anti-CTLA-4 antibody is administered Q8W.

10. The method of claim 7, wherein the activatable anti-CTLA-4 antibody is administered at a flat dose of 240, 600, 800, 1200 or 1600 mg.

11. The method of claim 10 wherein nivolumab is administered at a flat dose of 480 mg Q4W.

12. The method of claim 11 wherein Activatable Ipilimumab is administered at a flat dose of 600 mg Q4W.

13. The method of claim 11 wherein Activatable Ipilimumab is administered at a flat dose of 1200 mg Q8W.

14. The method of claim 7 wherein the cancer is selected from the group consisting of:
a. advanced renal cell carcinoma;
b. microsatellite instability-high (MSI-H) or mismatch repair deficient (dMMR) metastatic colorectal cancer;
c. melanoma;
d. non-small cell lung cancer (NSCLC);
e. malignant pleural mesothelioma; and
f. hepatocellular carcinoma.


SEQUENCE LISTING (27 sequences)

SEQ ID NO: 1 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 2 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 5 aa, Artificial Sequence
  CDR1 HC for Ipilimumab
SYTMH

SEQ ID NO: 4 - protein, 17 aa, Artificial Sequence
  CDR2 HC for Ipilimumab
FISYDGNNKYYADSVKG

SEQ ID NO: 5 - protein, 9 aa, Artificial Sequence
  CDR3 HC for Ipilimumab
TGWLGPFDY

SEQ ID NO: 6 - protein, 12 aa, Artificial Sequence
  CDR1 LC for Ipilimumab
RASQSVGSSYLA

SEQ ID NO: 7 - protein, 7 aa, Artificial Sequence
  CDR2 LC for Ipilimumab
GAFSRAT

SEQ ID NO: 8 - protein, 9 aa, Artificial Sequence
  CDR3 LC for Ipilimumab
QQYGSSPWT

SEQ ID NO: 9 - protein, 118 aa, Artificial Sequence
  Ipilimumab-VH
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 10 - protein, 329 aa, Artificial Sequence
  Human IgG1 constant HC
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 447 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 448 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 108 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 14 - protein, 107 aa, Artificial Sequence [sequence omitted]

SEQ ID NO: 15 - protein, 215 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 6 aa, Artificial Sequence
  Spacer
QGQSGS

SEQ ID NO: 17 - protein, 13 aa, Artificial Sequence
  Masking Moiety - YV39
CRTQLYGYNLCPY

SEQ ID NO: 18 - protein, 13 aa, Artificial Sequence
  Cleavable Moiety - 2001
ISSGLLSGRSDNH

SEQ ID NO: 19 - protein, 13 aa, Artificial Sequence
  Cleavable Moiety - 2011
ISSGLLSGRSDNP

SEQ ID NO: 20 - protein, 13 aa, Artificial Sequence
  Cleavable Moiety - 2012
ISSGLLSGRSANP

SEQ ID NO: 21 - protein, 146 aa, Artificial Sequence
  YV39-2001 VL
CRTQLYGYNLCPYGGGSSGGSISSGLLSGRSDNHGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 22 - protein, 146 aa, Artificial Sequence
  YV39-2011 VL
CRTQLYGYNLCPYGGGSSGGSISSGLLSGRSDNPGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 23 - protein, 146 aa, Artificial Sequence
  YV39-2012 VL
CRTQLYGYNLCPYGGGSSGGSISSGLLSGRSANPGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 24 - protein, 259 aa, Artificial Sequence
  Spacer - YV39-2011 LC
QGQSGSCRTQLYGYNLCPYGGGSSGGSISSGLLSGRSDNPGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 439 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC